(19) United States
(12) Plant Patent Application Publication
(10) Pub. No.: US 2018/0160605 P1
(43) Pub. Date: Jun. 7, 2018

(54) Sansevieria Plant Named 'SUPSAN1606'

(50) ;

(71) Applicant: Suphatchatwong Innovation Co., Ltd, Chonburi (TH)

(72) Inventor: Thumrong Suphachadiwong, Bangphra (TH)

(73) Assignee: Suphatchatwong Innovation Co., Ltd., Chonburi (TH)

(21) Appl. No.: 15/732,596

(22) Filed: Nov. 30, 2017

(30) Foreign Application Priority Data
Dec. 1, 2016 (QZ) 2016/3020

(51) Int. Cl. A01H 5/12

(52) U.S. Cl. PLT/382

(57) ABSTRACT

A new and distinct Sansevieria plant named ‘SUPSAN1606’ which is characterized by the combination of a relatively large plant size with an outward growth habit and very broad orbicular plant form, long, linear succulent foliage with an outward attitude and significant spacing between individual leaves, terete foliage with numerous shallow axial grooves and a deeper central channel, dark green foliage with inconspicuous mottled radial bands which are slightly darker green, and the stability of all characteristics from generation to generation.

### CROSS REFERENCE TO RELATED APPLICATIONS

This application claims priority to the Community Plant Variety Rights application number 2016/3020, filed Dec. 1, 2016, which is herein incorporated by reference.

Latin name of the genus and species: The Latin name of the genus and species of the novel variety disclosed herein is Sansevieria hybrid.

### Variety denomination:

The inventive variety of Sansevieria disclosed herein has been given the variety denomination ‘SUPSAN1606’.

### BACKGROUND OF THE INVENTION

Parentage: The Sansevieria variety ‘SUPSAN1606’ originated as a seedling selection from the controlled pollination of the proprietary seed parent, Sansevieria hybrid ‘MXJM1061’ (not patented), with the proprietary pollen parent, Sansevieria hybrid ‘MXJF1061’ (not patented). Both parents were developed by, and are owned by, the inventor and were never commercially released. Said crossing was conducted by the inventor in a small, netted greenhouse in Chonburi, Thailand in December of 2009. Seeds from said cross were harvested, then germinated, and the resulting seedlings were grown to a mature size in order to evaluate for a desirable combination of commercial characteristics. In April of 2012, one candidate plant was observed to exhibit a unique growth habit and leaf coloration. After confirmation of the distinctness and stability of the characteristics first observed, the inventor selected the new Sansevieria cultivar, ‘SUPSAN1606’, for commercial introduction.

Asexual Reproduction: Asexual reproduction of ‘SUPSAN1606’ by way of harvesting vegetative ground shoots was first initiated in April of 2012 at Chonburi, Thailand. Access to all plants was restricted, as plants were kept in a greenhouse not open to the public. Through subsequent propagation of vegetative ground shoots, five generations have been reproduced which have shown that the unique features of this cultivar are stable and reproduced true to type.

### SUMMARY OF THE INVENTION

The cultivar ‘SUPSAN1606’ has not been observed under all possible environmental conditions and the phenotype may vary somewhat with variations in environment such as temperature, day length, and light intensity, without, however, any variance in genotype. The following traits have been repeatedly observed and are determined to be the unique characteristics of ‘SUPSAN1606’. These characteristics in combination distinguish ‘SUPSAN1606’ as a new and distinct Sansevieria cultivar:
1. Sansevieria ‘SUPSAN1606’ is a relatively large plant with an outward growth habit and very broad orbicular plant form; and
2. Sansevieria ‘SUPSAN1606’ exhibits long, linear succulent foliage with an outward attitude and significant spacing between individual leaves; and
3. Sansevieria ‘SUPSAN1606’ exhibits terete foliage with numerous shallow axial grooves and a deeper central channel; and
4. Sansevieria ‘SUPSAN1606’ exhibits dark green foliage with inconspicuous mottled radial bands which are slightly darker green.

### BRIEF DESCRIPTION OF THE FIGURES

FIG. 1 illustrates, as nearly true as it is reasonably possible to make the same in color photographs of this type, an exemplary plant of ‘SUPSAN1606’ grown in a commercial greenhouse in Chonburi, Thailand. This plant is approximately 12 months old, shown planted in a 10 cm container.

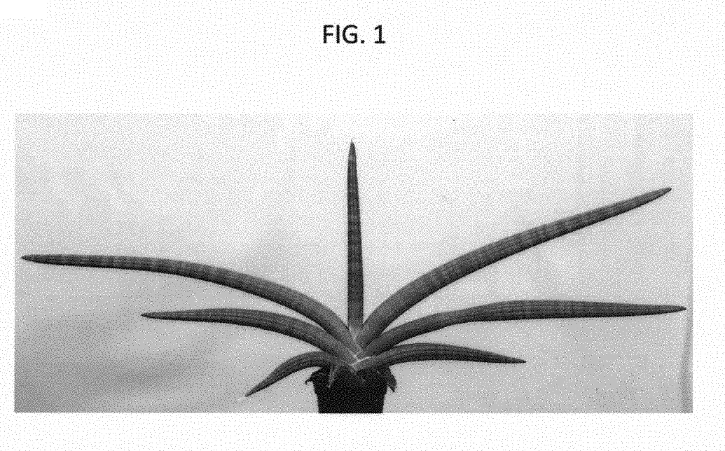

FIG. 2 illustrates, as nearly true as it is reasonably possible to make the same in color photographs of this type, the typical foliage of ‘SUPSAN1606’.

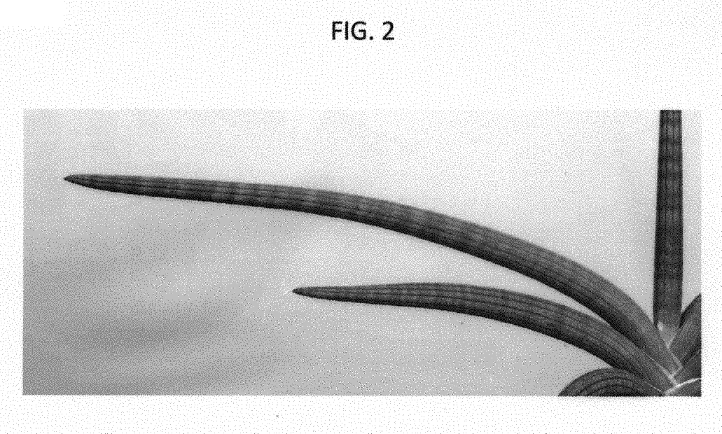

### BOTANICAL DESCRIPTION OF THE PLANT

The following observations and measurements, made in October of 2017, describe averages from a sample set of six specimens of 12 month old ‘SUPSAN1606’ plants grown in 10 cm containers, at a commercial greenhouse in Chonburi, Thailand. The plants were grown using conventional greenhouse production protocols for Sansevieria plants which consisted of overhead irrigation, 50% shade cloth, and no fertilizer. No photoperiodic or chemical treatments were given to the plants.

Those skilled in the art will appreciate that certain characteristics will vary with older or, conversely, with younger plants. ‘SUPSAN1606’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘SUPSAN1606’ and comparisons with the parents and most similar commercial variety of Sansevieria are provided below.
Plant description:
   Growth habit.—Monopodial perennial; equitant.
   Plant form.—Vertically flattened, very broad flattened orbicular.
   Average height.—32.5 cm.
   Plant spread.—24.4 cm at the narrowest point, and 90.0 cm at the widest point.
   Plant vigor.—Moderate.
   Growth rate.—Moderate.
   Propagation type.—Division of vegetative ground shoots; harvesting ground shoots from the mother plant.
   Propagation details.—The time needed to root a shoot division is approximately 2 to 3 months with temperatures ranging from approximately 17 to 40 degrees Celsius.
   Disease resistance.—Plants have not been observed to be susceptible or resistant to pathogens and pests common to Sansevieria.
   Environmental tolerances.—Adapt to, at least, USDA Zones 10 and 12 and temperatures as high as 40 degrees Celsius; moderate tolerance to rain; moderate to high tolerance to wind.
Root system:
   General.—Moderately dense and moderately branched rooting; roots are slightly fibrous.
   Distribution in the soil profile.—Shallow to moderately deep.
   Diameter of roots.—0.2 cm on average.
   Texture.—Smooth; no root hairs.
   Color.—Greyed-orange, nearest to RHS 165B.
Stem:
   Branching habit.—Monopodial, equitant; decurrent leaf bases form the stem.
   Number of primary (main) stems per plant.—One.
   Number of secondary (lateral) branches per plant.—None.
   Appearance and shape.—Stem is not visible; decurrent leaf bases form the stem.
   Length.—The decurrent leaf bases collectively have an average length of 6.2 cm.
   Diameter.—The decurrent leaf bases collectively have an average diameter of 2.3 cm.
   Internode length.—0.9 cm.
Foliage:
   Arrangement.—Distichous.
   Attachment.—Decurrent.
   Division.—Simple.
   Quantity.—7.
   Attitude.—At an average angle of 72.5 degrees to vertical.
   Lamina.—Dimensions — 46.7 cm long and 2.3 cm wide. Thickness — Approximately 2.4 cm. Shape of blade — Narrowly linear in outline. Cross-section — Terete with numerous shallow axial grooves and a deeper central channel. Aspect — Slightly curved downward. Apex — Narrowly acute with a papery tip, which is colored greyed-white, nearest to RHS 156C. Base — Decurrent. Margin — Leaves are terete so there are no margins, with the exception of the leaf base; leaf base margins are entire. Texture of upper surface — Glabrous; smooth; succulent. Texture of lower surface — Glabrous; smooth; succulent. Luster of the upper surface — Matte. Luster of the lower surface — Matte. Color — Juvenile foliage, upper surface — Nearest to in between green, RHS NN137A, and yellow-green, RHS 147A, with mottled radial bands which are colored nearest to a mixture of yellow-green, RHS 147B, and greyed-green, RHS 191B. Juvenile foliage, lower surface — Nearest to in between green, RHS NN137A, and yellow-green, RHS 147A, with mottled radial bands which are colored nearest to a mixture of yellow-green, RHS 147B, and greyed-green, RHS 191B; base is yellow-green, nearest to RHS 144C. Mature foliage, upper surface — Nearest to in between green, RHS 137B, and yellow-green, RHS 147A, with mottled radial bands which are colored yellow-green, nearest to a mixture of RHS 147B and 147C. Mature foliage, lower surface — Nearest to in between green, RHS 137B, and yellow-green, RHS 147A, with mottled radial bands which are colored yellow-green, nearest to a mixture of RHS 147B and 147C; margins of the decurrent base are papery and translucent and colored greyed-white, nearest to RHS 156A. Venation — Pattern — Parallel. Color, upper surface — Nearest to in between green, RHS 139A, and yellow-green, RHS 147A, but slightly darker. Color, lower surface — Nearest to in between green, RHS 139A, and yellow-green, RHS 147A, but slightly darker.
   Petiole.—No petioles present, leaves are decurrent.
Inflorescence: No flowering has been observed to date.
Comparisons with the parent plants: Plants of the new cultivar ‘SUPSAN1606’ differ from the seed parent, Sansevieria hybrid ‘MXJM1061’ (not patented), by the characteristics described in Table 1.

TABLE 1

| Characteristic | ‘SUPSAN1606’ | ‘MXJM1061’ |
|---|---|---|
| Plant size. | Larger than | Smaller than |
| | ‘MXJM1061’. | ‘SUPSAN1606'. |
| Foliage cross section. | Terete with numerous | Terete. |
| | shallow axial grooves and | |
| | a deeper central channel. | |
| Foliage thickness. | Thicker. | Thinner. |
| Foliage texture. | Less smooth than | Smoother than |
| | ‘MXJM1061’. | ‘SUPSAN1606’. |
| General coloration of the | Darker green closer to | Lighter green. |
| foliage. | greyed-green. | |
| General coloration of the | Darker green. | Lighter green. |
| leaf venation. | | |

Plants of the new cultivar ‘SUPSAN1606’ differ from the pollen parent, Sansevieria hybrid ‘MXJF1061’ (not patented), by the characteristics described in Table 2:

TABLE 2

| Characteristic | ‘SUPSAN1606’ | ‘MXJF1061’ |
|---|---|---|
| Plant size. | Larger than | Smaller than |
| | ‘MXJF1061’. | ‘SUPSAN1606’. |
| Foliage length. | Longer than | Shorter than |
| | ‘MXJF1061’. | ‘SUPSAN1606’. |
| Foliage thickness. | Thicker. | Thinner. |
| Foliage texture. | Smoother than | Less smooth than |
| | ‘MXJF1061’. | ‘SUPSAN1606’. |
| General coloration of the | Darker green closer to | Greyed-green. |
| foliage. | greyed-green. | |
| General coloration of the | Lighter green. | Darker green. |
| leaf venation. | | |

Comparison with the most similar Sansevieria cultivar known to the inventor: Plants of the new cultivar ‘SUPSAN1606’ are most similar to the commercial cultivar, Sansevieria cylindrica ‘SAN201202’ (U.S. Plant Pat. No. 24,457). A comparison of ‘SUPSAN1606’ with ‘SAN201202’ is described in Table 3.

TABLE 3

| Characteristic | ‘SUPSAN1606’ | ‘SAN201202’ |
|---|---|---|
| Growth habit. | Less compact than | More compact than |
| | ‘SANS201202’. | ‘SUPSAN1606’. |
| Foliage attitude. | More outward; greater | More upright; individual |
| | spacing between | leaves are held more |
| | individual leaves. | tightly together. |
| Leaf aspect. | Slightly curved | Generally straight. |
| | downward. | |
| Foliage apex. | Narrowly acute. | Acute. |
| General coloration of | Darker green closer to | Lighter green. |
| the foliage. | greyed-green. | |
| Prominence of the | Less prominent green | More prominent green |
| color pattern | mottled radial bands. | mottled radial bands. |
| on the foliage. | | |

That which is claimed is:

1. A new and distinct variety of Sansevieria plant named ‘SUPSAN1606’, substantially as described and illustrated herein.
